(19) United States
(12) Patent Application Publication
(10) Pub. No.: US 2022/0160600 A1
(43) Pub. Date: May 26, 2022

(54) SUNSCREEN COMPOSITIONS

(71) Applicant: BEIERSDORF AG, Hamburg (DE)

(72) Inventors: Yongyi ZHANG, New Providence, NJ (US); Sherilee BACKMAN, Morganville, NJ (US)

(21) Appl. No.: 17/310,241

(22) PCT Filed: Jan. 30, 2020
(86) PCT No.: PCT/US2020/015876

§ 371 Date: Jul. 27, 2021

Related U.S. Application Data
(60) Provisional application No. 62/800,152, filed on Feb. 1, 2019.

(51) Int. Cl. A61K 8/37; A61K 8/06; A61K 8/60; A61K 8/81; A61K 8/34; A61K 8/35; A61K 8/40; A61Q 17/04

(52) U.S. Cl. CPC A61K 8/37; A61K 8/062; A61K 8/602; A61K 8/8147; A61Q 17/04; A61K 8/8182; A61K 8/342; A61K 8/35; A61K 8/40; A61K 8/8188

(57) ABSTRACT

Discloses are compositions which are suitable for use as sunscreen lotions and creams. The compositions are high oil load emulsions which exhibit both a quick-break effect, as well as physical stability at different temperature levels. Also disclosed are methods of manufacturing and using such compositions.

### FIELD

The present disclosure relates generally to sun protection compositions, and more specifically to sun protection compositions made up of emulsions having a quick break effect and physical stability.

### BACKGROUND

A sunscreen product with US FDA approved organic type sunscreen actives is often considered greasy and heavy due to the nature of the sunscreen actives. The skin feel needs to be improved for a lotion aiming at beauty sector consumers. What is needed in the art are formulations that improve the initial skin feel, skin feel during drying time, and final skin feel. Moreover the formulations need to maintain physical stability at different temperature levels.

### BRIEF SUMMARY

Provided herein are compositions, suitable for use as sunscreen lotions and creams, that improve the initial skin feel, skin feel during drying time, and final skin feel. In some aspects, the compositions provided are a quick-break high oil load emulsions that maintain superior physical stability at different temperature levels.

In certain aspects, the composition comprises at least one organic active agent and a plurality of emulsifiers. In some embodiments, one of the emulsifiers comprises polyacrylate. In some embodiments, another of the emulsifiers comprises at least one glucoside and at least one alcohol. In certain embodiments of the foregoing, the composition is an oil-in-water emulsion. In some variations, the composition is an oil-in-water quick break emulsion system. In certain variations, the at least one organic active agent is at least one organic sunscreen active agent, and the composition is a sunscreen composition.

In other aspects, provided is a method of applying the sunscreen composition onto skin, scalp, and/or hair of a human.

In yet other aspects, provided is an article of manufacture comprising: a container comprising any of the compositions described herein; and a label containing instructions for use of the composition.

### DESCRIPTION OF THE FIGURES

The present application can be understood by reference to the following description taken in conjunction with the accompanying figures.

FIG. 1 is a 1-dimensional perceptual map comparing performance during manipulation (Firmness, Stickiness, Peaking, Integrity of Shape & Thickness) with performance during rubout (Wetness, Spreadability, Slipperiness, Oily, Absorbency).

### DETAILED DESCRIPTION

The following description sets forth exemplary methods, parameters and the like. It should be recognized, however, that such description is not intended as a limitation on the scope of the present disclosure but is instead provided as a description of exemplary embodiments.

Provided herein are compositions with formulation aesthetics that target beauty sector consumers, as well as sunscreen sector consumers. When organic sunscreen actives are used in a formulation, the skin feel generally needs to be improved for a lotion aiming at beauty sector consumers. In some aspects, the compositions herein are suitable for use as sunscreen lotions, and incorporate ingredients carefully selected to improve the initial skin feel, skin feel during drying time, and final skin feel. In addition to the choice of ingredients, a quick-break emulsion technology is employed to provide consumers with an extraordinary skin sensation and reveal a good cooling effect.

In some aspects, the compositions provided are made up of at least one organic active agent and a plurality of emulsifiers. In some variations, two or more emulsifiers are present in the compositions. One emulsifier comprises polyacrylate, and another emulsifier comprises at least one glucoside and at least one alcohol. When the at least one organic active agent is at least one organic sunscreen active agent, the compositions provided herein are suitable for use as sunscreens.

The various ingredients of the compositions, as well as properties, methods of manufacturing and using the compositions are further described below.

### Emulsion System

In some embodiments, the compositions are oil-in-water (O/W) emulsions with quick break properties. In certain embodiments, the compositions are quick-break, high oil load emulsions.

In some variations, the compositions have an oil load of at least 5%, at least 10%, at least 15%, at least 20%, at least 25%, or at least 30%; or between 5% and 50%, between 10% and 50%, between 15% and 50%, between 20% and 50%, between 25% and 50%, between 30% and 50%, between 35% and 50%, between 40% and 50%, between 5% and 45%, between 10% and 45%, between 15% and 45%, between 20% and 45%, between 25% and 45%, between 30% and 45%, between 5% and 40%, between 10% and 40%, between 15% and 40%, between 20% and 40%, between 25% and 40%, between 30% and 40%, based on the total weight of the composition.

Various emulsifiers are used in the compositions.

Polyacrylate Emulsifiers

In one embodiment, one of the emulsifiers comprises polyacrylate.

In some variations, the polyacrylate emulsifier comprises a crosspolymer of a sulfonic acid acrylamide monomer or a salt thereof, and an alkyl methacrylate monomer or a salt thereof. In certain variations, the polyacrylate emulsifier comprises a crosspolymer of an ammonium salt of a sulfonic acid acrylamide monomer, and an alkyl methacrylate monomer.

In other variations, the polyacrylate emulsifier comprises a crosspolymer of: a monomer of formula (M1) and a monomer of formula (M2), wherein:
the monomer of formula (M1) is:

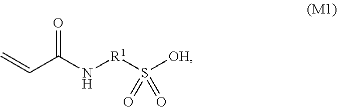

or a salt thereof, wherein R¹ is alkylene;
the monomer of formula (M2) is:

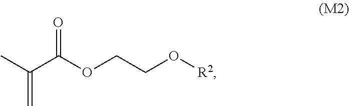

or a salt thereof, wherein R² is alkyl.

In certain variations of the foregoing, the monomer of formula (M1) is an ammonium salt. In certain variations of the foregoing, R¹ is a branched alkylene. In one variation, R¹ is —C₁₋₁₀ alkylene. In certain variations, R² is C₁₅₋₃₀ alkyl.

Suitable polyacrylate emulsifiers may include, for example, ammonium acryloyldimethyltaurate/beheneth-25 methacrylate crosspolymer (e.g., Aristoflex HMB); acrylates/palmeth-25 acrylate copolymer (e.g., Rheostyl E-Light); sodium acrylate/acryloyldimethyltaurate/dimethylacrylamide crosspolymer and isohexadecane and polysorbate 60 (e.g., Simulgel SMS 88); ammonium acryloyldimethyltaurate/carboxyethyl acrylate crosspolymer (e.g., Aristoflex TAC); acrylate/C10-30 alkyl acrylate crosspolymer (e.g., Pemulen TR-1 and TR-2, Protamer Z-21); acrylates copolymer (e.g., Emul 33); carbomer (e.g. Ashland 981 Carbomer); and acrylic acid/vinyl pyrrolidone crosspolymer (e.g., UltraThix P-100).

A combination of the foregoing polyacrylate emulsifiers may also be used in the compositions described herein.

Glucoside/Alcohol Emulsifiers

In another embodiment, another of the emulsifiers comprises at least one glucoside and at least one alcohol. In one embodiment, the alcohol is a fatty alcohol. In certain variations, the alcohol is a C₈₊ fatty alcohol. In another variation, the alcohol is a C₁₂ to C₂₅ fatty alcohol. In some variations, the length of the alcohol and the glucoside is the same or similar.

In some variations, the glucoside/alcohol emulsifier comprises at least one glucoside and at least one fatty alcohol. In certain variations, the glucoside/alcohol emulsifier comprises one, two or three glucosides. In another variation that may be combined with the foregoing, the glucoside/alcohol emulsifier comprises one, two, or three fatty alcohols.

In other variations, at least one glucoside is a glucoside of formula (G1):

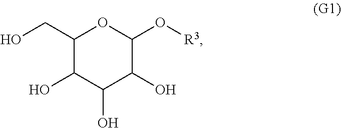

or a salt thereof, wherein R³ is alkyl.

In one variation, the glucoside of formula (G1) is a glucoside of formula (G1a):

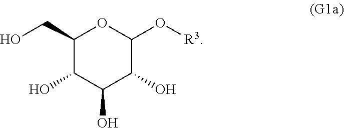

In certain variations of the foregoing, R³ is a C₈₊ alkyl. In another variation, R³ is a C₁₂ to C₂₅ alkyl.

In some embodiments, the glucoside/alcohol emulsifiers may include additional components, such as seed oil and stearates.

Suitable glucoside/alcohol emulsifiers may include, for example, arachidyl alcohol and behenyl alcohol and arachidyl glucoside (e.g., Montanov 202); cetearyl glucoside and cetearyl alcohol (e.g., Montanov 68, SpecSufc SC-M68); coco-glucoside and coconut alcohol (e.g. Montanov S); myristyl alcohol and myristyl glucoside (e.g., Montanov 14); cetearyl alcohol and coco-glucoside (e.g., Montanov 82); C14-22 alcohols and C12-20 alkyl glucoside (e.g., Montanov L); cetearyl glucoside and sorbitan olivate and cetearyl alcohol (e.g., Beautyderm K10); cetearyl alcohol and polysorbate 60 and cetearyl glucoside (e.g., Uniox Cristal); hydroxyethyl acrylate/sodium acryloyldimethyl taurate copolymer and cetearyl alcohol and arachidyl alcohol and coco-glucoside and behenyl alcohol and arachidyl glucoside (e.g., Simplynov 365); cetearyl alcohol and glyceryl stearate and sorbitan stearate and cetearyl glucoside (e.g., Emulpharma Coreosome); and Cannabis sativa seed oil or other oil and C12-18 alkyl glucoside and glyceryl stearate and cetearyl alcohol and stearic acid (e.g., AE EmulsiPure Hempseed).

It is understood that any description of the polyacrylate emulsifiers may be combined with any descriptions of the glycoside/alcohol emulsifiers the same as if each and every combination were individually listed. For example, in one variation, at least two of the emulsifiers in the compositions are: (i) ammonium acryloyldimethyltaurate/beheneth-25 methacrylate crosspolymer, and (ii) arachidyl alcohol, behenyl alcohol and arachidyl glucoside.

### Active Agents

The compositions provided herein comprise at least one active agent. The active agents are typically skincare active agents. In some variations, skincare active agents may include materials regarded as acceptable for use as active skin-protecting ingredients. A skincare active agent may include, for example, skin protectant and/or anti-aging agent. Approval by a regulatory agency may sometimes be required for inclusion of active agents in formulations intended for human contact, including for example, sunscreen active ingredients or skin protectant ingredients.

The active agents present in the composition may differ based on the purpose of the composition. For example, a sunscreen composition comprises sunscreen active agents. In some variations, the active agent(s) is/are organic active agent(s).

Any suitable sunscreen active agents may be part of the sunscreen composition. Approved sunscreen active agents in the United States and elsewhere include, for example, para-aminobenzoic acid, avobenzone, cinoxate, dioxybenzone, homosalate, menthyl anthranilate, octocrylene, octyl methoxycinnamate, octyl salicylate, oxybenzone, 2-ethylhexyl 4-(dimethylamino)benzoate (e.g., Padimate O), phenylbenzimidazole sulfonic acid, octisalate, sulisobenzone, and trolamine salicylate. Several other sunscreen active ingredients are accepted for use in other countries. Examples from outside the U.S. include Tinosorb M, Tinosorb S, Uvinul T-150, UVA sorb HEB, Uvinul A Plus, Neo Heliopan AP, and Neo Heliopan MBC.

In certain variations, the compositions comprise at least one organic active agent selected from avobenzone, octocrylene, octisalate, and homosalate, or any combination thereof. In one variation, the compositions comprise avobenzone, octocrylene, octisalate, and homosalate.

In certain embodiments, in addition to the skin-active ingredients already described, the compositions provided comprise one or more additional skin-active ingredients, such as a humectant and moisturizing ingredients, an anti-aging agent, a depigmenting agent, an anti-wrinkle agent, or an agent that treats oily skin. Additional active agents may include, for example, adenosine, hyaluronic acid, lanolin, citric acid, malic acid, lactic acid, tartaric acid, salicylic acid, a vitamin, a retinoid, retinol, retinoic acid, a carotenoid, an amino acid, a protein, an enzyme, and a coenzyme.

It is typical to use combinations of two or more skincare active agents in a formulation. The amount of skincare active agent or agents may be present in an amount that is consistent with the guidelines of the FDA or other regulatory bodies. The use of a combination of active agents is especially true for sunscreen formulations to achieve higher levels of ultraviolet absorption or to provide useful absorption over a wider range of ultraviolet wavelengths than can be the case with a single active component. Preferably, the sunscreen active agent or agents is present in an amount that is consistent with the FDA sunscreen monograph for sunscreen active agent or agents that are believed to provide the requisite SPF in accordance with the FDA monograph for such sunscreens. Other skin care active agents may include, for example, sunless tanning active agents, skin protectant active agent emollients, and insect repelling agents.

In some variations, the active agent(s) are present in the compositions herein in an amount of from 10% to 40%, from 20% to 40%, or from 20% to 35% by weight of the composition.

### Other Ingredients

In some embodiments, the compositions herein include additional ingredients, such as solvents, film formers, SPF boosters, emulsification and stabilization system components, skin feel enhancers, excipients, vitamins, and preservation system components.

Solvents

In certain embodiments, one or more solvents are present in the compositions described herein. In some variations, the solvent comprises water. In one embodiment, from 40% to 70%, based on the total weight of the composition, of water is present. In another embodiment, from 40% to 70%, based on the total weight of the composition, of water and alcohol are present.

In some variations, little to no alcohol is present in the compositions. In certain variations, less than 15%, less than 10%, less than 5%, less than 1%, based on the total weight of the composition, of alcohol is present. In one variation, the composition has no alcohol. In variations of the composition where alcohol is present, suitable alcohols, such as ethanol, may be used.

Film Formers

In certain embodiments, one or more film formers are present in the compositions described herein. In some variations, a combination of film formers is used.

In some embodiments, the one or more film formers are present in an amount of not more than about 7.5%, about 5%, about 4%, about 3%, about 2%, about 1%, about 0.5%, about 0.25%, or about 0.1% by weight. In certain embodiments, the one or more film formers are present in an amount of about 3 wt % based on the total weight of the sunscreen formulation.

In some variations, the one or more film formers are present in a dry matter content ranging from 0.1% to 60%, from 0.5% to 40%, or from 1% to 30% by weight relative to the total weight of the composition.

In one embodiment, the film formers are a polymer capable of forming, on its own or in the presence of a film-forming aid, a continuous and adherent film on a support, for instance on the keratinous materials.

In some variations, the film formers used are capable of forming a hydrophobic film. In certain variations, the film formers are polymers capable of forming a hydrophobic film having a solubility in water at 25° C. of less than 1% by weight.

In other variations, the film formers may be synthetic polymers of the free-radical type or of the polycondensate type, polymers of natural origin and mixtures thereof. Free-radical film-forming polymers include polymers obtained by polymerization of monomers with for instance, ethylenic unsaturation, each monomer being capable of homopolymerizing (in contrast to polycondensates).

In other variations, the film formers may be of the free-radical type, for example, vinyl polymers or copolymers. Examples include acrylic polymers. In one variation, the vinyl film-forming polymers may result from the polymerization of ethylenically unsaturated monomers having at least one acid group and/or esters of these acid monomers and/or amides of these acid monomers. In another variation, as a monomer carrying an acid group, there may be used C.B-ethylenic unsaturated carboxylic acids such as acrylic acid, methacrylic acid, crotonic acid, maleic acid and itaconic acid. For example, (meth)acrylic acid and itaconic acid may be used. In yet another variation, the esters of acid monomers may be chosen from the esters of (meth)acrylic acid (also called (meth)acrylates), for example alkyl, such as C—Co, for instance C—C, alkyl, (meth)acrylates, aryl, such as Co—Co aryl, (meth)acrylates, hydroxyalkyl, for instance C-C hydroxyalkyl, (meth)acrylates. Examples of alkyl (meth)acrylates may include methyl methacrylate, ethyl methacrylate, butyl methacrylate, isobutyl methacrylate, 2-ethyl hexyl methacrylate, lauryl methacrylate and cyclohexyl methacrylate. Examples of hydroxyalkyl (meth)acrylates may include hydroxyethyl acrylate, 2-hydroxypropyl acrylate, hydroxyethyl methacrylate and 2-hydroxypropyl methacrylate. Examples of aryl (meth)acrylates may include benzyl acrylate and phenyl acrylate. In certain variations the alkyl group of the esters may be either fluorinated or perfluorinated (e.g., some or all of the hydrogen atoms of the alkyl group may be substituted with fluorine atoms). Examples of amides of the acid monomers may include (meth)acrylamides, such as N-alkyl(meth)acrylamides, and for instance, of a C—C alkyl. Examples of N-alkyl(meth)acrylamides may include N-ethylacrylamide, N-t-butylacrylamide, N-t-octylacrylamide and N-undecylacrylamide.

In some variations, the vinyl film formers may also result from the homopolymerization or copolymerization of monomers chosen from vinyl esters and styrene monomers. For example, these monomers may be polymerized with acid monomers and/or their esters and/or their amides, such as those mentioned above. Examples of vinyl esters may include vinyl acetate, vinyl neodecanoate, vinyl pivalate, vinyl benzoate and vinyl t-butylbenzoate. Examples of styrene monomers may include styrene and alpha-methylstyrene. In other variations, any acrylic and vinyl monomers (including the monomers modified by a silicone chain) may be used.

In other variations, the film formers are film-forming polycondensates, such as polyurethanes, polyesters, polyester amides, polyamides, epoxy ester resins, and polyureas. In one variation, the polyamides include, for example, polyamide-8. In another variation, the polyurethanes may be chosen from anionic, cationic, nonionic and/or amphoteric polyurethanes, polyurethane-acrylics, polyurethane-polyvinylpyrrolidones, polyester-polyurethanes, polyether-polyurethanes, polyureas, and polyurea-polyurethanes. In certain variations, the polyesters may be obtained by polycondensation of dicarboxylic acids with polyols, such as diols. In yet other variations, the dicarboxylic acids may be aliphatic, alicyclic and/or aromatic. Suitable acids may include, for example, oxalic acid, malonic acid, dimethylmalonic acid, succinic acid, glutaric acid, adipic acid, pimelic acid, 2.2-dimethylglutaric acid, azelaic acid, suberic acid, sebacic acid, fumaric acid, maleic acid, itaconic acid, phthalic acid, dodecanedioic acid, 1.3-cyclohexanedicarboxylic acid, 1,4-cyclohexanedicarboxylic acid, isophthalic acid, terephthalic acid, 2.5-norbornanedicarboxylic acid, diglycolic acid, thiodipropionic acid, 2.5-naphthalenedicarboxylic acid and 2.6-naphthalenedicarboxylic acid. These dicarboxylic acid monomers described herein may be used alone or in combination with at least two dicarboxylic acid monomers. Examples of monomers that may be used are phthalic acid, isophthalic acid and terephthalic acid. In other variations, the diols may be chosen from aliphatic, alicyclic and/or aromatic diols. For example, the diols may be chosen from: ethylene glycol, diethylene glycol, triethylene glycol, 1,3-propanediol, cyclohexanedimethanol and 4-butanediol. Examples of polyols may include glycerol, pentaerythritol, sorbitol, and trimethylolpropane.

In some variations, the polyester amides may be obtained in a manner similar to the polyesters, by polycondensation of diacids with diamines or amino alcohols. As diamines, there may be used ethylenediamine, hexamethylenediamine, meta- and/or para-phenylenediamine. As aminoalcohols, monoethanolamine may be used. In certain variations, the polyester may, in addition, comprise at least one monomer carrying at least one —SOM group, wherein M is chosen from hydrogen atoms, ammonium ions and metal ions (such as for example Na, Li, K, Mg, Ca, Cu, Fe ions). In other variations, the at least one monomer may be a bifunctional aromatic monomer comprising such an —SOM group. In certain variations, the aromatic ring of the bifunctional aromatic monomer carrying an —SOM group as described above may be chosen, for example, from benzene, naphthalene, anthracene, diphenyl, oxydiphenyl, sulphonyl diphenyl and methylenediphenyl rings, sulphoisophthalic acid, sulphoterephthalic acid, sulphophthalic acid, and 4-sulphonaphthalene-2,7-dicarboxylic acid.

In other variations, copolymers may be used. In certain variations, the copolymers are based on isophthalate or sulphoisophthalate. In certain variations, the copolymers may be obtained by condensation of diethylene glycol, cyclohexanedimethanol, isophthalic acid and sulphoisophthalic acid. In certain variations, the optionally modified polymers of natural origin may be chosen from shellac resin, sandarac gum, dammars, elemis, copals, cellulosic polymers and mixtures thereof.

In some embodiments, the film formers may be present in the form of particles in aqueous dispersion, generally known as latex or pseudolatex. The techniques for preparing these dispersions are well known to persons skilled in the art.

In other variations, the film formers are an aqueous dispersion of film-forming polymers. In certain variations, the film formers are dispersions of polymers resulting from the free-radical polymerization of one or more free radical monomers inside and/or partly at the surface, of preexisting particles of at least one polymer chosen from polyurethanes, polyureas, polyesters, polyesteramides and/or alkyls. These polymers are generally called hybrid polymers.

In other variations, the film formers may include a water-soluble polymer which is present in the aqueous phase of the composition in solubilized form. Examples of film-forming water-soluble polymers may include proteins. In one variation, the proteins are of plant origin (such as wheat or soya bean proteins), proteins of animal origin (such as keratins, for example keratin hydrolysates and sulphonic keratins), as well as anionic, cationic, amphoteric or nonionic polymers of chitin or chitosan. In other variations, the polymers are cellulose polymers such as hydroxyethyl cellulose, hydroxypropyl cellulose, methyl cellulose, ethyl hydroxyethyl cellulose, carboxymethyl cellulose, and quaternized derivatives of cellulose. In yet other variations, the polymers are acrylic polymers or copolymers such as poly acrylates or polymethacrylates. In yet other variations, the polymers are vinyl polymers, such as polyvinylpyrrolidones, copolymers of methyl vinyl ether and maleic anhydride, copolymers of vinyl acetate and crotonic acid, copolymers of vinylpyrrolidone and vinyl acetate; copolymers of vinylpyrrolidone and caprolactam; polyvinyl alcohol: In other variations, the polymers are optionally modified polymers of natural origin, such as gum arabic, guar gum, xanthan derivatives, karaya gum, glycoaminoglycans, hyaluronic acid and its derivatives; shellac resin, sandarac gum, dammars, elemis, copals; alginates and carrageenans; deoxyribonucleic acid; and muccopolysaccharides such as chondroitin sulphates.

In one embodiment, the film formers are selected from vinyl polymers, vinyl copolymers (such as acrylic polymers or acrylates), fluorinated or perfluorinated acrylates, acrylamides, vinyl esters, styrene esters, silicone-modified vinyl polymers, vinyl copolymers, acrylates, acrylamides, other vinyl esters, styrene esters, polyurethanes, polyesters, polyester amides, polyamides, epoxy ester resins, polyureas, optionally modified polymers of natural origin, shellac resin, sandarac gum, dammars, elemis, copals, cellulosic polymers, hydrolyzed starches, latexes, abietates, hybrid polymers, proteins, anionic, cationic, amphoteric or nonionic polymers of chitin or chitosan, cellulose polymers, and pullulan.

In other variations, the film formers are selected from anionic, cationic, nonionic and/or amphoteric polyurethanes, polyurethane-acrylics, polyurethane-polyvinylpyrrolidones, polyester-polyurethanes, polyether-polyurethanes, polyureas, and polyurea-polyurethanes. In one variation, the film formers are diols.

In another variation, the film formers are selected from wheat or Soya bean proteins; proteins of animal origin such as keratins, for example keratin hydrolysates and sulphonic keratins.

In yet another variation, the film formers are selected from hydroxyethyl cellulose, hydroxypropyl cellulose, methyl cellulose, ethyl hydroxyethyl cellulose, carboxymethyl cellulose, and quaternized derivatives of cellulose.

In yet another variation, the film formers are selected from bis-octyldodecyl dimer dilinoleate/propanediol copolymer and octyldodecyl/glyceryl hydroxy stearate dilinoleate dimethicone copolymer.

SPF Boosters

In certain embodiments, one or more SPF boosters are present in the compositions described herein. In some variations, a combination of SPF boosters is used.

In some embodiments, the one or more SPF boosters are present in an amount of between 0.1% and 10%, or between 0.1% and 8%; or about 10%, about 9%, about 8%, about 7%, about 6%, about 5%, about 4%, about 3%, about 2%, about 1%, about 0.5%, about 0.2%, or about 0.1% by weight of the total composition.

Suitable SPF boosters may include butyloctyl salicylate, ethylhexyl methoxycrylene, styrene/acrylates copolymer, styrene/acrylates copolymer (and) acrylates copolymer, glycogen, acrylates/methacryloyloxyethyl phosphate copolymer, dimethyl capramide, neopentyl glycol diethylhexanoate (and) neopentyl glycol diisostearate, daucus carota sative (carrot) root extract (and) helianthus annuus (sunflower) seed oil, argania spinosa kernel oil (and) tocopheryl acetate (and) bisabolol, PVP (and) VP/Eicosene copolymer, VP/eicosene copolymer, hydrophobically modified starches, silica beads, PMMA beads, borosilicate beads, polyurethane beads, diatomaceous earth, betonite and hectorite clays and any combination thereof.

Emulsifiers and Stabilizers

In certain embodiments, one or more emulsifiers are present in the compositions described herein. In other embodiments, one or more humectants and other stabilizers are also present in the compositions described herein. In some variations, a combination of emulsifiers, humectants and other stabilizers is used.

In some variations, the compositions further comprise a silicone-based emulsifier. Suitable silicone-based emulsifiers may include cetyl PEG/PPG 10/1 Dimethicone, cetyl diglyceryl tris(trimethylsiloxy) silylethyl dimethicone, sorbitan olivate, steareth-20, steareth-2, steareth-21, methyl glucose dioleate, bis-PEG/PPG-14/14 dimethicone (and) dimethicone, dimethicone (and) dimethicone/vinyl dimethicone crosspolymer (and) PEG-10 dimethicone, cyclopentasiloxane (and) dimethicone (and) dimethicone/vinyl dimethicone crosspolymer (and) PEG-10 dimethicone, polyglyceryl-4 isostearate (and) coco-caprylate/caprate (and) hectorite, polyglyceryl-2 dipolyhydroxystearate, lauryl PEG-8 dimethicone, PEG-10 dimethicone, dimethicone (and) caprylyl dimethicone ethoxy glucoside, cylcopentasiloxane (and) caprylyl dimethicone ethoxy glucoside, dimethicone (and) PEG/PPG-18/18 dimethicone, cyclopentasiloxane (and) PEG/PPG-20/15 dimethicone, cyclopentasiloxane (and) PEG-10 dimethicone (and) Bentonite (and) distearyldimonium chloride, dimethicone (and) PEG/PPG-18/18 dimethicone, PEG-8 dimethicone, PEG/PPG-19/19 dimethicone (and) C13-16 isoparaffin (and) C10-13 isoparaffin, mineral oil (and) PEG-15/lauryl dimethicone crosspolymer, bis-isobutyl PEG/PPG-10/7/dimethicone copolymer, sorbitan olivate, cylcopentasiloxane (and) PEG/PPG-18/18 dimethicone and any combination thereof.

Suitable humectants may include moisturizing humectants (such as glycerin, hydroxyethyl urea, betaine, sodium PCA, sodium-L-lactate and propanediol), antimicrobial potentiating humectants (such as pentylene glycol, and hexanediol), humectant solvents (such as hexanediol, PEG-4, 8, dipropylene glycol, pentylene glycol, propanediol, butylene glycol, 2-methyl-1,3-propanediol and propylene glycol) and natural based humectants (such as glycerin, pentylene glycol, propanediol, sodium PCA, sodium-L-Lactate and betaine). In other variations, the humectants may include glyceryl esters (such as glycereth-26).

Other suitable stabilizers may include chlorides (such as sodium chloride, potassium chloride, and magnesium chloride), carbonates (such as sodium carbonate, potassium carbonate, magnesium carbonate and propylene carbonate), sulfates (such as sodium sulfate, zinc sulfate, and magnesium sulfate), silica, polymeric thickeners, natural gums, olefin resins, waxes, and any combination thereof.

In some variations, a photostabilizer is used. In certain embodiments, the photostabilizers may also boost SPF.

Skin Feel Enhancers

In certain embodiments, one or more skin feel enhancers are present in the compositions described herein. In some variations, a combination of the skin feel enhancers described herein is used.

Suitable skin feel enhancers may include elastomers and silicones. In some embodiments, the skin feel enhancers are dimethicone fluids. In some variations, the skin feel enhancers are silicon elastomer blends, or polydimethylsiloxane fluids. In certain variations, the skin feel enhancers may be silicone crosspolymers, dimethicone crosspolymers dimethicone/vinyl dimethicone crosspolymers polysilicone-modified silicones, hydrocarbon/silicone crosspolymers, hydrocarbon/dimethicone crosspolymers, hydrocarbon crosspolymers, alkyl/silicone crosspolymers, alkyl/dimethicone crosspolymers, or alkyl crosspolymers. In other variations, the skin feel enhancers are dimethicones, cyclic siloxanes, linear silicones, organofunctional silicones, or organofunctional polydimethylsiloxanes. In other variations, the skin feel enhancers are squalene or hemisqualene. In yet other variations, the skin feel enhancers are isododecane.

In some variations, the skin feel enhancers used in the compositions described herein helps to mitigate product drag, imparts dry cushion during and after rubout, as well as help to anchor the sunscreen film on the skin, and improve product spreadability and dry time.

Excipients

In certain embodiments, the composition further comprises one or more excipients. Suitable excipients may include compounds that can help to improve skin feel, viscosity, stability, SPF boosting capability, and cost. Other suitable excipients may include, for example, esters (such as ethylhexyl isononanoate, C12-15 alkyl benzoate, isopropyl palmitate, diisopropyl adipate, diethylhexyl carbonate), and beeswax.

Vitamins

In certain embodiments, the compositions may further comprise one or more vitamins. For example, in some variations, vitamin E and/or vitamin C may be present in the compositions described herein. In some embodiments, vitamin E and/or vitamin C may be present at about 2%, about 1%, about 0.5%, about 0.25%, about 0.1%, about 0.5%, about 0.25%, or about 0.01% by weight.

Preservation System Components

In certain embodiments, the compositions may further comprise one or more preservation system components. Such preservation system components may include ingredients that provide additional protection against bacterium growth, such as biostatic agents. Suitable preservation system components may include, for example, hydroxyacetophenone and 1,2-hexanediol.

Other Ingredients

In certain embodiments, the compositions may further comprise botanicals, such as plant extracts.

In some variations, the compositions are formulated as lotions or creams, including sunscreen lotions or sunscreen creams. In one variation, the composition is a gel lotion.

In some embodiments, the compositions herein are free of one or more or all of oxybenzone, octinoxate, paraben, phenoxyethanol, colorant, phthalates and formaldehyde donors. In one variation, the composition has less than 0.1, 0.01, or 0.001 wt % of oxybenzone, octinoxate, paraben, phenoxyethanol, colorant, phthalates and formaldehyde donors.

### Composition Properties

The compositions described herein have one or more of the following properties.

Quick Break Effect

In some embodiments, the compositions provided exhibit a quick break effect. For example, in one embodiment, the composition is an oil-in-water quick break emulsion system. In some variations, quick break refers to the instant release of the main part of the water phase in the composition when applied on top of skin.

Physical Stability

In other embodiments, the compositions provided are physically stable. In some variations the compositions exhibit physical stability over a range of temperatures. For example, in certain embodiments, physical stability include is based one or more, or all, of the following conditions:

Condition 1. One month at 50° C. with visual observation;

Condition 2. Three months at 40° C. with visual observation and viscosity check;

Condition 3. Three months at 5° C. with visual observation;

Condition 4. Six months at room temperature with visual observation and viscosity check;

Condition 5. Freeze thaw test with visual observation: 48 hours at −20° C. and 48 hours at room temperature for 4 cycles; and

Condition 6. Transportation test with visual observation: 48 hours at −20° C. and 48 hours at 60° C. for 3 cycles.

Other Properties

In some variations, liquid crystal structures are present in the composition.

In other variations, the sunscreen formulations described herein have a Sun Protection Factor (SPF), measured according to the method prescribed by the U.S. Food and Drug Administration, of at least 15, at least 20, at least 25, at least 30, at least 35, at least 40, at least 45, at least 50, at least 55, or at least 60; or between 15 and 75, or between 15 and 70, between 20 and 65, between 50 and 70, or between 55 and 60. In one variation, the sunscreen formulations described herein have a SPF of about 15, 30 or 50.

In other variations, the sunscreen formulations described herein have a UVA Protection Factor (UVAPF), measured according to the method prescribed by the U.S. Food and Drug Administration, of at least about 16, at least about 17, at least about 18, at least about 19, at least about 20, at least about 21, or at least about 22. In some embodiments, the sunscreen formulations have a UVAPF in a range from about 16 to about 20, or about 16 to about 17. In one variation that may be combined with any of the foregoing variations, the sunscreen formulation has a SPF of 50.

In yet other variations, the UVAPF is higher than one third of the total SPF value, and meets the critical wavelength of higher than 370 nm.

In yet another variation, the sunscreen formulations described herein have a water resistance, measured according to the method prescribed by the U.S. Food and Drug Administration, of up to about 80 minutes, for example, about 40 minutes, about 50 minutes, about 60 minutes, about 70 minutes or about 80 mins. In other variations, the sunscreen formulations described herein have a water resistance of at least 40 minutes. In some embodiments, the sunscreen formulations have a water resistance in a range from about 40 to about 80 minutes, or from about 40 to about 60 minutes.

In another variation, the sunscreen formulations described herein are tear-free.

In yet other variations, the compositions herein have an ultra-light hydrating formula, a powerful water splash which can immediately transform cream to water, and provide an immediate surge of hydration.

### Methods of Making the Compositions

In certain aspects, provided herein is a method of manufacturing the compositions described herein.

In some embodiments, the method comprises: combining and mixing the oil phase ingredients, while heating to a temperature that is about 5 degrees above the melting point of the highest melting point oil phase ingredient; adding polyacrylate with continuous mixing until well-dispersed; separately combining and mixing the aqueous phase ingredients until uniform; adding the hot oil phase to the hot water phase with high shear mixing; removing heat and mixing until uniform, while the batch was cooled; and adding and mixing one or more additional ingredients and water to reach the final weight. In some variations of the foregoing, the silicone and isododecane may be added as part of the first step involving the combining and mixing of the oil phase ingredients, or post emulsification.

In some variations, the method comprises:
a) combining the organic active agent(s) with other oil-soluble ingredients to form a first mixture, wherein the first mixture comprises an oil phase;
b) mixing and heating the first mixture to a first temperature;
c) adding polyacrylate (such as ammonium acryloyldimethyltaurate/beheneth-25 methacrylate crosspolymer) to the heated first mixture with continuous mixing;
d) combining the water-soluble ingredients to form a second mixture, wherein the second mixture comprises an aqueous phase;
e) mixing and heating the second mixture;
f) applying homogenization if necessary;
g) mixing and heating the mixture in step (f) to a second temperature to form the hot aqueous phase;
h) adding the hot oil phase to the hot water phase with high shear mixing; and
i) cooling the mixture in step (h) to a third temperature;
j) post adding ingredients such as preservation system components, fragrances, skin feel enhancers etc., followed by water and optionally alcohol, to the mixture in step (i) while mixing to produce the composition.

In some variations, the first temperature is between about 180° F. and about 200° F., or between about 185° F. and about 195° F.

In some variations, the second temperature is between about 150° F. and about 180° F.

In some variations, the third temperature is below about 140° F. or below about 104° F.

### Use of the Compositions

In some aspects, provided is a method of applying the compositions described herein. In some embodiments, provided is a method comprising applying any of the compositions described herein onto skin, scalp, and/or hair of a human. In one variation, provided is a method comprising applying any of the compositions onto the face and/or body of a human.

In other aspects, provided is a system to dispense the compositions described herein. In one aspect, provided is an article of manufacture, such as a container comprising any of the compositions described herein, and a label containing instructions for use of the composition. In some embodiments, the container is a tube, containing the composition formulated as a lotion (e.g., tubed lotion).

### EXAMPLES

The following Examples are merely illustrative and are not meant to limit any aspects of the present disclosure in any way.

### Example 1

### Exemplary Sunscreen Formulation (“Formulation A”)

This example describes an exemplary sunscreen formulation of the disclosure in Table 1 below, and methods of manufacturing thereof.

TABLE 1
Formulation A

| Ingredient Description | Weight Percent w/w |
|---|---|
| Part A Ingredients | |
| Avobenzone | 3 |
| Octocrylene | 9 |
| Homosalate | 9 |
| Octisalate | 4.5 |
| Other Oil-Soluble Ingredients (*) | 1-20 |
| Arachidyl Alcohol and Behenyl Alcohol | 0.1-5 |
| and Arachidyl Glucoside | |
| Part B Ingredients | |
| Ammonium Acryloyldimethyltaurate/ | 0.1-5 |
| Beheneth-25 | |
| Methacrylate Crosspolymer | |
| Part C Ingredients | |
| Purified Water | 30-70 |
| Other Water-Soluble Ingredient (**) | 0.1-20 |
| Part D Ingredients | |
| Additional Ingredients post-added (***) | 0.1-15 |
| Part E Ingredients | |
| Purified Water | Q.S. |
| Total | 100.00 |

Other Oil-Soluble Ingredients (*) in Part A may include, for example, anti-oxidants, emollients, film formers, SPF boosters, skin feel enhancers, and other oil-soluble excipients. Other Water-Soluble Ingredient (**) in Part C may include, for example, humectants, thickeners, solvents, SPF boosters, preservatives, and other water-soluble ingredients. Additional Ingredients post-added (***) in Part D may include, for example, solvents, fragrances, skin feel enhancers, and SPF boosters.

### Manufacturing Method

The formulation in Table 1 above was prepared as follows: In a container large enough to hold the oil phase, all the ingredients of Part A (oil phase) were added and mixed while heating the phase to about 5 degrees Fahrenheit above the melting point of the highest melting point ingredient in Part A (e.g., 185° F. to 194° F.). After the oil phase was observed to be within the target temperature range, ammonium acryloyldimethyltaurate/beheneth-25 methacrylate crosspolymer of Part B was added with continuous mixing until well-dispersed. In a container large enough to hold the entire batch, the ingredients of Part C (aqueous phase) were combined with mixing or homogenization until uniform with heating to about the same temperature range as Part A. The hot oil phase was gradually added to the hot water phase with high shear mixing. Heat was removed, and mixing continued until uniform while the batch was cooled to a temperature ranging from 86° F. to 140° F. The ingredients of Part D were gradually added with mixing, such as alcohol, fragrances, skin feel enhancers etc. Water of Part E was finally added to reach the final weight, and mixed well to form the final composition.

### Quick Break Effect Testing

A sample of Formulation A was applied and spread over a small area on human skin. Instant water release and cooling effect was felt on the skin, indicating that the formulation exhibited a quick break effect.

### Stability Testing

Stability of Formulation A was tested at various temperatures, between −20 and 60° C., including repeated freeze thaw testing and subject the sample to long term storage at elevated temperature to justify at least a two year shelf life. The samples were inspected by visual observation and or rheological and other analytical testing. Formulation A was observed to be physically and chemically stable based on the conditions set forth above.

### Example 2

### Emulsion System Screen

This example compares various emulsion systems with respect to emulsion performance, specifically quick break and physical stability.

Various formulations were prepared based on the emulsifiers and emulsion type specified in Table 2 below. The formulations were manufactured in accordance with the protocol set forth in Example 1 above. Quick break effect and physical stability were also tested in accordance with the protocols set forth in Example 1 above.

TABLE 2

| | Co- | Emulsion | |
|---|---|---|---|
| Primary Emulsifier | Emulsifier | Type | Emulsion Performance |
| Acrylates/C10-30 Alkyl Acrylate | n/a | O/W | No quick break observed. |
| Crosspolymer | | | |
| Lauryl PEG-10 | n/a | W/O | No quick break observed. |
| Tris(Trimethylsiloxy)silylethyl | | | |
| Dimethicone | | | |
| Cetyl PEG/PPG-10/1 Dimethicone | n/a | W/O | No quick break observed. |
| Ammonium | n/a | O/W | Quick break observed. |
| Acryloyldimethyltaurate/Beheneth- | | | F/T failed. |
| 25 Methacrylate Crosspolymer | | | |
| Ammonium | Beeswax | O/W | F/T improved stability. |
| Acryloyldimethyltaurate/Beheneth- | | | Less quick break |
| 25 Methacrylate Crosspolymer | | | observed. |
| Ammonium | Reduced | O/W | F/T failed. |
| Acryloyldimethyltaurate/Beheneth- | amount of | | |
| 25 Methacrylate Crosspolymer | Beeswax | | |
| Ammonium | Cetyl Alcohol | O/W | F/T failed. |
| Acryloyldimethyltaurate/Beheneth- | | | |
| 25 Methacrylate Crosspolymer | | | |
| Ammonium | Cetyl Alcohol | O/W | F/T failed. |
| Acryloyldimethyltaurate/Beheneth- | and Beeswax | | |
| 25 Methacrylate Crosspolymer | | | |
| Ammonium | Polyglyceryl-6 | O/W | No quick break observed. |
| Acryloyldimethyltaurate/Beheneth- | Stearate (and) | | Improved F/T stability. |
| 25 Methacrylate Crosspolymer | Polyglyceryl-6 | | |
| | Behenate | | |
| Increased amount of Ammonium | n/a | O/W | F/T failed. |
| Acryloyldimethyltaurate/Beheneth- | | | |
| 25 Methacrylate Crosspolymer | | | |
| Ammonium | Arachidyl | O/W | Stable with quick break |
| Acryloyldimethyltaurate/Beheneth- | Alcohol (and) | | effect observed. |
| 25 Methacrylate Crosspolymer | Behenyl | | |
| | Alcohol (and) | | |
| | Arachidyl | | |
| | Glucoside | | |

“O/W” refers to an oil-in-water emulsion, in which the external or continuous phase is water. “W/O” refers to a water-in-oil emulsion, in which the external or continuous phase is oil.

The results in Table 2 above show that the combination of Aristoflex HMB (an exemplary polyacrylate emulsifier) and Montanov 202 (an exemplary glucoside/alcohol emulsifier) provided both the desired quick break effect and physical stability.

### Example 3

### Comparative Study of Sensory Attributes of Sunscreen Lotion Formulations

This example compares the sensory attributes of various commercially available sunscreen lotions and Formulation A.

In a consumer panel study, Formulation A was found to possess distinct sensorial attributes distinguishing it from various commercially available sunscreen lotions.

In the study, Formulation A was distinguished as exhibiting better perceived sensorial attributes of wetness, quickbreak, spreadability, absorption, and slipperiness, and less stickiness and stiffness, than various commercially available sunscreen lotions.

Study Design

The study design involved monadic assessments of sunscreen formulations in a randomized and balanced complete block design and estimate mean value for each sensory attribute for each product. A trained consumer panel performed all assessments. Consumer panelists were selected on their ability to detect and discriminate differences in visual and tactile properties. All panelists were validated and extensively trained in evaluation of lotions, sprays, creams, and related product forms.

Attribute intensity was rated on a universal 101 point intensity scale using 1-point increments. Panelists were trained to use the scale in a similar way across panelists and across samples. Use of a universal scale allowed attributes to be compared in intensity to one another, (e.g. comparing intensity of slippery feel to intensity of sticky feel), as well as for comparison of samples within and across studies and products having shared attributes.

All data was collected from the individual respondents, and evaluations were replicated.

### Products Tested

Formulation A was compared with several existing commercial sunscreen lotions. All sunscreen lotions meet an SPF 50 rating.

### Results

Product sensory profiles (individual data means) were used to develop perceptual maps. Perceptual maps of the samples were developed to better understand the relationships among the attributes that define the sensory space of the sample category tested, and where the products fall in that space.

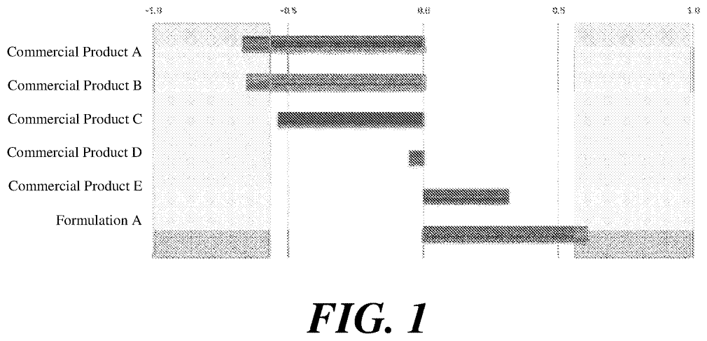

With reference to FIG. 1, samples in the shaded areas are considered well-defined by the dimension. Products that fell within the shaded area to the left of the map were observed to have the following properties: When products were manipulated, they were firmer, stickier, generated higher peaks and maintained their shape. They also felt thicker during rubout. Products that fell within the shaded area to the right of the map were observed to have the following properties: When rubbed into the skin, the product felt more wet, oily and slippery, was easier to spread, but took longer to absorb.

1.-33. (canceled)

34. A composition, wherein the composition is an oil-in-water emulsion which comprises:
at least one organic active agent; and
at least two emulsifiers, at least one of the emulsifiers being (i) a first emulsifier comprising polyacrylate, and at least one other of the emulsifiers being (ii) a second emulsifier comprising at least one glucoside and at least one alcohol.

35. The composition of claim 34, wherein the composition is an oil-in-water quick break emulsion system.

36. The composition of claim 34, wherein the composition has an oil load of at least 5%, based on a total weight of the composition.

37. The composition of claim 34, wherein liquid crystal structures are present in the composition.

38. The composition of claim 34, wherein the first emulsifier comprises a crosspolymer of a sulfonic acid acrylamide monomer or a salt thereof, and an alkyl methacrylate monomer or a salt thereof.

39. The composition of claim 34, wherein the first emulsifier comprises a crosspolymer of an ammonium salt of a sulfonic acid acrylamide monomer, and an alkyl methacrylate monomer.

40. The composition of claim 34, wherein the first emulsifier comprises a crosspolymer of: a monomer of formula (M1) and a monomer of formula (M2), wherein:
the monomer of formula (M1) is: 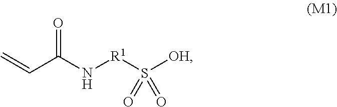
or a salt thereof, wherein R¹ is alkylene;
the monomer of formula (M2) is: 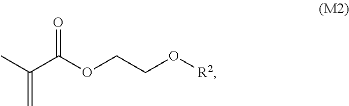
or a salt thereof, wherein R² is alkyl.

41. The composition of claim 40, wherein the monomer of formula (M1) is an ammonium salt.

42. The composition of claim 40, wherein R¹ is a branched alkylene and/or is C₁₋₁₀ alkylene.

43. The composition of claim 40, wherein R² is C₁₅₋₃₀ alkyl.

44. The composition of claim 34, wherein the second emulsifier comprises at least one glucoside and at least one fatty alcohol.

45. The composition of claim 34, wherein the second emulsifier comprises two fatty alcohols.

46. The composition of claim 34, wherein at least one glucoside is a glucoside of formula (G1): 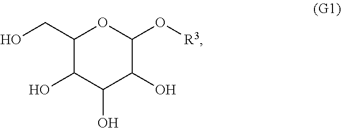
or a salt thereof, wherein R³ is alkyl.

47. The composition of claim 46, wherein R³ is a C₈₊ alkyl.

48. The composition of claim 34, wherein at least two of the emulsifiers are (i) ammonium acryloyldimethyltaurate/beheneth-25 methacrylate crosspolymer, and (ii) arachidyl alcohol, behenyl alcohol and arachidyl glucoside.

49. The composition of claim 34, wherein the first emulsifier comprises:
ammonium acryloyldimethyltaurate/beheneth-25 methacrylate crosspolymer;
acrylates/palmeth-25 acrylate copolymer;
sodium acrylate/acryloyldimethyltaurate/dimethylacrylamide crosspolymer and isohexadecane and polysorbate 60;
ammonium acryloyldimethyltaurate/carboxyethyl acrylate crosspolymer;
acrylate/C10-30 alkyl acrylate crosspolymer;
acrylates copolymer;
carbomer; or
acrylic acid/vinyl pyrrolidone crosspolymer.

50. The composition of claim 34, wherein the second emulsifier comprises:
arachidyl alcohol and behenyl alcohol and arachidyl glucoside;
cetearyl glucoside and cetearyl alcohol;
coco-glucoside and coconut alcohol;
myristyl alcohol and myristyl glucoside;
cetearyl alcohol and coco-glucoside;
C14-22 alcohols and C12-20 alkyl glucoside;
cetearyl glucoside and sorbitan olivate and cetearyl alcohol;
cetearyl alcohol and polysorbate 60 and cetearyl glucoside;
hydroxyethyl acrylate/sodium acryloyldimethyl taurate copolymer and cetearyl alcohol and arachidyl alcohol and coco-glucoside and behenyl alcohol and arachidyl glucoside;
cetearyl alcohol and glyceryl stearate and sorbitan stearate and cetearyl glucoside; or
Cannabis sativa seed oil or other oil and C12-18 alkyl glucoside and glyceryl stearate and cetearyl alcohol and stearic acid.

51. The composition of claim 34, wherein the at least one organic active agent is at least one organic sunscreen active agent.

52. The composition of claim 34, wherein the at least one organic active agent comprises one or more of avobenzone, octocrylene, octisalate, homosalate.

53. A method of protecting human skin, scalp and/or hair from harmful sun radiation, wherein the method comprises applying the composition of claim 34 onto skin, scalp, and/or hair of a human.
